(19) United States
(12) Patent Application Publication
(10) Pub. No.: US 2004/0076670 A1
(43) Pub. Date: Apr. 22, 2004

(54) Process for producing pulverulent active substance formulations with compressible fluids

(76) Inventors: Bernd Klinksiek, Bergisch Gladbach (DE); Lars Obendorf, Koln (DE); Rainer Bellinghausen, Odenthal (DE); Marcus Eichmann, Dusseldorf (DE)

Correspondence Address: WILLIAM GERSTENZANG, NORRIS, MCLAUGHLIN & MARCUS, P.A., 220 EAST 42ND STREET, 30TH FLOOR, NEW YORK, NY, 10017, US

(21) Appl. No.: 10/679,123

(22) Filed: Oct. 3, 2003

(30) Foreign Application Priority Data
Oct. 18, 2002 (DE) 10248619.0

(51) Int. Cl. A61K 9/24

(52) U.S. Cl. 424/471

(57) ABSTRACT

Pulverulent active substance formulations composed of

at least one active substance which is solid at room temperature,

at least one dispersant B),

polyvinyl alcohol and

optionally, additives C),

the individual active substance particles being enveloped by a coat of polyvinyl alcohol, being present in the amorphous state, and having diameter in the nanometer range, a process for producing said formulations using compressible fluids, use of said active substance formulations for the application of the active substance they comprise, and apparatus for producing the pulverulent active substance formulations.

This invention pertains to an innovative dispersing process for producing fine active substance particles using CO₂ and for producing emulsions from high-viscosity oils.

### BACKGROUND OF THE INVENTION

The process is a development on the melt dispersing process described in the DE 10 151 392.5, WO 03/034822. In melt dispersing processes, a solids suspension is heated to above the melting point of the suspended solids, then finely emulsified and thereafter cooled rapidly to give a fine, amorphous dispersion.

In the production of dispersions with compressible fluids, melting and emulsification are possible at lower temperatures, and hence less aggressively as far as the product is concerned. This effect can probably be attributed to the fact that the compressible fluid dissolves in the active substance, lowers the melting temperature and decreases the viscosity of the melt.

The CO₂ based process is therefore an alternative worth considering for temperature-sensitive substances for which conventional melt dispersing cannot be employed.

The use of CO₂ also allows fine emulsification of high-viscosity oils. The reason for this is probably the suitability of compressible fluids as solvents for the oil, so that the high viscosity is reduced.

From the literature (e.g. Chem. Eng. Proc. 2000, 39, 19-28, Chem. Ing. Tech. 1997, 69, 298-311) a host of processes are known for producing fine particles with compressible fluids.

Essentially a distinction is made between groups of processes, in which

1. the supercritical fluid acts as an antisolvent and the particles are generated by precipitation/crystallization from a substance solution (GAS: Gas Anti Solvent, PCA: Precipitation with a Compressed Fluid Antisolvent, SEDS:

Solution Enchanced Dispersion of Solids),

2. the supercritical fluid is the solvent and the particles form when the fluid is depressurized into a container (RAPID EXPANSION OF A SUPERCRITICAL SOLUTION —RESS),

3. the supercritical fluid is dissolved in substance melts or suspensions and fine particles are formed when the substance/fluid mixture is depressurized into a vessel (PGSS: Particle Generation from Gas Saturated Solution, CPCSP:

Continuous Powder Coating Spraying Process, CPF: Concentrated Powder Form).

The way in which the processes work can be traced back to phenomena such as solvent properties of compressed or supercritical fluids, lowering of melting points, viscosity reduction, increased diffusion coefficients and mass transfer coefficients, etc. Since in the majority of cases combinations of the phenomena are involved, it is not always possible to categorize the processes unambiguously. Marr and Gamse (Chem. Eng. Proc. 2000, 39, 19-28) give a general overview of the use of supercritical fluids in industrial processes. The production of microparticles with compressed gases is examined by Bungert et al. (Chem. Ing Tech. 1997, 69, 298-311).

In the case of the PARTICLES FROM GAS SATURATED SOLUTIONS (PGSS) process (EP 744 992 A1) a solid is melted under the gas pressure of a compressible fluid, which dissolves in the solid, and is depressurized through a nozzle into a spraying tower. Micronization takes place as a result of the explosive effect of the compressible fluid dissolved in the melt. The size of the active particles is in the region of 10 μm.

In the process of Mura (EP 661 091 A1) a solid is first of all melted individually and then dispersed in supercritical fluids. Micronization takes place, again, by depressurizing into a spraying tower.

In the process of Odell (U.S. Pat. No. 05 487 965) a suspension of particulate solids in a carrier liquid is produced to start with. This suspension is then dispersed in turn in a supercritical fluid and is subsequently depressurized for micronization.

### SUMMARY OF THE INVENTION

Novel pulverulent active substance formulations have now been found which are comprised of

at least one active substance A) which is solid at room temperature,

at least one dispersant B),

a coating material E), and

optionally additives C),

the individual active substance particles being coated with a coat of coating material E), in particular polyvinyl alcohol, being present in particular in an amorphous state and having an average diameter in the region of not more than 1 μm.

It has further been found that the pulverulent active substance formulations of the invention can be produced by

a) suspending at least one active substance A) which is solid at room temperature, at least one dispersant B), and optionally additives C) in an aqueous phase, in particular at room temperature,

b) adding at least one compressible fluid D) in the supercritical state under pressure to the suspension formed in a),

c) heating the mixture formed in b) until the solid components it comprises are liquefied,

d) homogenizing the resultant dispersion and then adding an aqueous solution of coating material E), especially of polyvinyl alcohol, alone or in a mixture with other coating material E), and optionally additives C),

e) suddenly depressurizing the dispersion and subjecting it, in particular simultaneously, to drying, preferably to spray drying or freeze drying, with particular preference to spray drying.

Finally it has been found that the pulverulent active substance formulations of the invention are especially suitable for the application of the active substances they comprise.

### DETAILED DESCRIPTION OF THE INVENTION

For the purposes of the invention an amorphous state is one for which on analysis by means of DSC no phase transformation can be recognized or for which x-ray diffraction analyses reveal substantially no crystal structure.

Suitable fluids D) for the purposes of the invention are, in particular, fluids selected from the group consisting of hydrocarbons having 1 to 6 carbon atoms, especially methane, ethane, propane, butane, pentane, n-hexane, i-hexane, carbon dioxide, Freons® Perfluorocarbons, nitrogen, noble gases, gaseous oxides, such as N₂O, CO₂, ammonia, alcohols having 1 to 4 carbon atoms, especially methanol, ethanol, isopropanol, n-propanol, butanol, halogenated hydrocarbons, or mixtures of the aforementioned substances.

It is extremely surprising that the pulverulent active substance formulations of the invention are substantially more stable than the existing preparations constitutionally closest to them, which are obtainable by melt dispersing, but in which the individual particles are not encapsulated. Another reason for the unexpectedness of the stability of the formulations of the invention is that it would have been expected that the polyvinyl alcohol coat would dissolve in the water and that the active substance A) would then recrystallize. Contrary to these expectations, however, this effect did not occur.

The pulverulent active substance formulations of the invention are also notable for a number of advantages. For instance, the active substance content is very high as compared with corresponding prior art formulations. This means that just a small amount of formulation is sufficient to provide the desired quantity of active component. Another advantage is that the pulverulent active substance formulations of the invention can be redispersed readily prior to use and that the bioavailability of the active components remains at the high level achieved following production. It is favorable, finally, that the thermal load on the active substances A) during the production of the formulations is kept low.

Suitable active substances A) present in the pulverulent formulations of the invention are active pharmaceutical substances, active agrochemical substances, vitamins, carotenoids and aromas which are solid at room temperature (25° C.).

Examples of carotenoids which can be used are the known, available, natural or synthetic representatives of this class of compound, examples being carotene, lycopene, bixin, zeaxanthin, ctryptoxanthin, citranaxanthin, lutein, canthaxanthin, astaxanthin, β-apo-4′-carotenal, β-apo-8′-carotenal, β-apo-12′-carotene, β-apo-8′-carotenoic acid, and esters of hydroxy- or carboxy-containing compounds of this group, examples being lower alkyl esters; preferably methyl and ethyl esters. Particular preference is given to the industrially available representatives, such as β-carotene, canthaxanthin, β-apo-8′-carotenal and β-apo-8′-carotenoic esters.

It is likewise possible to use retinoids, examples being all-trans-retinoic acid, 13-cis-retinoic acid and the esters and amides of this acid. Compounds of this kind which can be used are described by D. L. Newton, W. R. Henderson and M. B. Sporn in Cancer Research 40, 3413-3425.

Examples that may be mentioned of active pharmaceutical substances A) are ibuprofen, clotrimazole, fluconazole, indoxacarb, acetylsalicylic acid and ciprofloxazin.

By active agrochemical substances A) in the present context are meant all substances customary for plant treatment whose melting point is above 20° C. With preference mention may be made of fungicides, bactericides, insecticides, acaricides, nematicides, molluscicides, herbicides and plant growth regulators.

Examples of fungicides which may be mentioned are:

2-anilino-4-methyl-6-cyclopropylpyrimidine; 2′, 6′-dibromo-2-methyl-4′-trifluoromethoxy-4′-trifluoromethyl-1,3-thiazole-5-carboxanilide; 2,6-dichloro-N-(4-trifluoromethylbenzyl)benzamide; (E)-2-methoximino-N-methyl-2-(2-phenoxyphenyl)acetamide; 8-hydroxyquinoline sulfate; methyl (E)-2-{2-[6-(2-cyanophenoxy)pyrimidine-4-yloxy]phenyl}-3-methoxyacrylate; methyl (E)-methoximino[alpha-(o-tolyloxy)-o-tolyl]acetate; 2-phenylphenol (OPP), ampropylfos, anilazin, azaconazole, benalaxyl, benodanil, benomyl, binapacryl, biphenyl, bitertanol, blasticidin-S, bromuconazole, bupirimate, buthiobate, calcium polysulfide, captafol, captan, carbendazim, carboxin, chinomethionat (quinomethionate), chloroneb, chloropicrin, chlorothalonil, chlozolinate, cufraneb, cymoxanil, cyproconazole, cyprofuram, carpropamid, dichlorophen, diclobutrazole, dichlofluanid, diclomezin, dicloran, diethofencarb, difenoconazole, dimethirimol, dimethomorph, diniconazole, dinocap, diphenylamine, dipyrithion, ditalimfos, dithianon, dodine, drazoxolon, epoxyconazole, ethirimol, etridiazole, fenarimol, fenbuconazole, fenfuram, fenitropan, fenpiclonil, fentin acetate, fentin hydroxide, ferbam, ferimzone, fluazinam, fludioxonil, fluoromide, fluquinconazole, flusilazole, flusulfamide, flutolanil, flutriafol, folpet, fosetyl-aluminium, fthalide, fuberidazole, furalaxyl, furmecyclox, fenhexamid, guazatine, hexachlorobenzene, hexaconazole, hymexazol, imazalil, imibenconazole, iminoctadin, iprobenfos (IBP), iprodione, isoprothiolane, iprovalicarb, kasugamycin, mancopper, mancozeb, maneb, mepanipyrim, mepronil, metalaxyl, metconazole, methasulfocarb, methfuroxam, metiram, metsulfovax, myclobutanil, nickel dimethyldithiocarbamate, nitrothal-isopropyl, nuarimol, ofurace, oxadixyl, oxamocarb, oxycarboxin, pefurazoate, penconazole, pencycuron, phosdiphen, pimaricin, piperalin, polyoxin, probenazole, prochloraz, procymidone, propamocarb, propiconazole, propineb, pyrazophos, pyrifenox, pyrimethanil, pyroquilon, quintozene (PCNB), quinoxyfen, tebuconazole, tecloftalam, tecnazen, tetraconazole, thiabendazole, thicyofen, thiophanate-methyl, thiram, tolclophos-methyl, tolylfluanid, triadimefon, triadimenol, triazoxide, trichlamid, tricyclazole, triflumizole, triforine, triticonazole, trifloxystrobin validamycin A, vinclozolin, zineb, ziram, 2-[2-(1-chloro-cyclopropyl)-3-(2-chlorophenyl)-2-hydroxypropyl]-2,4-dihydro-[1,2,4]-triazole-3-thione 3-(1-[2-(4-[2-chlorophenoxy)-5-fluoropyrimid-6-yloxy)-phenyl]-1-(methoximino)-methyl)-5,6-dihydro-1,4,2,-dioxazine and 2-(2-[6-(3-chloro-2-methylphenoxy)-5-fluoropyrimid-4-yloxy]-phenyl)-2-methoximino-N-methyl-acetamide.

Examples of bactericides which may be mentioned are:

bronopol, dichlorophen, nitrapyrin, octhilinone, furancarboxylic acid, oxytetracyclin, probenazole, tecloftalam.

Examples of insecticides, acaricides and nematicides which may be mentioned are:

abamectin, acephate, acrinathrin, alanycarb, aldicarb, alphamethrin, amitraz, avermectin, AZ 60541, azadirachtin, azinphos A, azinphos M, azocyclotin, 4-bromo-2-(4-chlorophenyl)-1-(ethoxymethyl)-5-(trifluoromethyl)-1H-pyrrole-3-carbonitrile, bendiocarb, benfuracarb, bensultap, betacyfluthrin, bifenthrin, BPMC, brofenprox, bromophos A, bufencarb, buprofezin, butocarboxin, butylpyridaben, cadusafos, carbaryl, carbofuran, carbophenothion, carbosulfan, cartap, chloethocarb, chloretoxyfos, chlorfluazuron, chlormephos, N-[(6-chloro-3-pyridinyl)-methyl]-N′-cyano-N-methyl-ethanimidamide, chlorpyrifos, chlorpyrifos M, cis-resmethrin, clocythrin, clofentezine, cyanophos, cycloprothrin, cyfluthrin, cyhalothrin, cyhexatin, cypermethrin, cyromazine, deltamethrin, demeton-M, demeton-S, demeton-S-methyl, diafenthiuron, diazinon, dichlofenthion, dichlorvos, dicliphos, dicrotophos, diethion, diflubenzuron, dimethoate, dimethylvinphos, dioxathion, emamectin, esfenvalerate, ethiofencarb, ethofenprox, ethoprophos, fenamiphos, fenazaquin, fenbutatin oxide, fenobucarb, fenothiocarb, fenoxycarb, fenpropathrin, fenpyrad, fenpyroximate, fenvalerate, fipronil, fluazuron, flucycloxuron, flucythrinate, flufenoxuron, flufenprox, fluvalinate, fonophos, formothion, fosthiazate, fubfenprox, furathiocarb, HCH, heptenophos, hexaflumuron, hexythiazox, imidacloprid, iprobenfos, isazophos, isofenphos, isoprocarb, isoxathion, ivermectin, lambda-cyhalothrin, lufenuron, mecarbam, mevinphos, mesulfenphos, metaldehyde, methacrifos, methamidophos, methidathion, methiocarb, methomyl, metolcarb, milbemectin, monocrotophos, moxidectin, naled, NC 184, nitenpyram, oxamyl, oxydeprofos, permethrin, phosalone, phosmet, phosphamidon, pirimicarb, profenophos, promecarb, propaphos, propoxur, prothiophos, prothoate, pymetrozine, pyrachlophos, pyridaphenthion, pyresmethrin, pyrethrum, pyridaben, pyrimidifen, pyriproxifen, quinalphos, salithion, sebufos, silafluofen, sulfotep, tebufenozide, tebufenpyrad, tebupirimiphos, teflubenzuron, tefluthrin, temephos, terbam, tetrachlorvinphos, thiacloprid, thiafenox, thiamethoxam, thiodicarb, thiofanox, thiomethon, thuringiensin, tralomethrin, transfluthrin, triarathene, triazuron, trichlorfon, triflumuron, trimethacarb, vamidothion, XMC, xylylcarb, zetamethrin.

Examples of molluscicides which may be mentioned are metaldehyde and methiocarb.

Examples of herbicides which may be mentioned are:

Anilides, such as, for example, diflufenican and propanil; arylcarboxylic acids, such as, for example, dichloropicolinic acid, dicamba and picloram; aryloxyalkanoic acids, such as, for example, 2,4-D, 2,4-DB, 2,4-DP, fluroxypyr, MCPA, MCPP and triclopyr; aryloxy-phenoxy-alkanoates, such as, for example, diclofop-methyl, fenoxaprop-ethyl, haloxyfop-methyl and quizalofop-ethyl; azinones, such as, for example, chloridazon and norflurazon; carbamates, such as, for example, chlorpropham, desmedipham, phenmedipham and propham; chloroacetanilides, such as, for example, alachlor, metazachlor, pretilachlor and propachlor; dinitroanilines, such as, for example, oryzalin, pendimethalin and trifluralin; diphenyl ethers, such as, for example, acifluorfen, bifenox, fluoroglycofen, fomesafen, halosafen, lactofen and oxyfluorfen; ureas, such as, for example, chlortoluron, diuron, fluometuron, isoproturon, linuron and methabenzthiazuron; hydroxylamines, such as, for example, alloxydim, clethodim, cycloxydim, sethoxydim and tralkoxydim; imidazolinones, such as, for example, imazethapyr, imazamethabenz, imazapyr and imazaquin; nitriles, such as, for example, bromoxynil, dichlobenil and ioxynil; oxyacetamides, such as, for example, mefenacet; sulfonylureas, such as, for example, amidosulfuron, bensulfuron-methyl, chlorimuron-ethyl, chlorsulfuron, cinosulfuron, metsulfuron-methyl, nicosulfuron, primisulfuron, pyrazosulfuron-ethyl, thifensulfuron-methyl, triasulfuron and tribenuron-methyl; thiolcarbamates, such as, for example, butylate, cycloate, diallate, EPTC, esprocarb, molinate, prosulfocarb and triallate; triazines, such as, for example, atrazine, cyanazine, simazine, simetryn, terbutryne and terbutylazine; triazinones such as, for example, hexazinon, metamitron and metribuzin; others, such as, for example, aminotriazole, benfuresate, bentazone, cinmethylin, clomazone, clopyralid, difenzoquat, dithiopyr, ethofumesate, fluorochloridone, glufosinate, glyphosate, isoxaben, pyridate, quinchlorac, quinmerac, sulfosate and tridiphane. 4-Amino-N-(1,1-dimethylethyl)-4,5-dihydro-3-(1-methylethyl)-5-oxo-1H-1,2,4-triazole-1-carboxamide and 2-((((4,5-dihydro-4-methyl-5-oxo-3-propoxy-1H-1,2,4-triazol-1-yl)carbonyl)amino)sulfonyl)-methyl benzoate may also be mentioned.

Examples of plant growth regulators which may be mentioned are chlorocholine chloride and ethephon.

Dispersants B) suitable for the purposes of the invention include all customary nonionogenic, anionic, cationic and zwitterionic substances, having the desired surface-active properties, that are normally used in such formulations. These substances include reaction products of fatty acids, fatty acid esters, fatty alcohols, fatty amines, alkylphenols or alkylarylphenols with ethylene oxide and/or propylene oxide, and also their sulphuric esters, phosphoric monoesters and phosphoric diesters, and also reaction products of ethylene oxide with propylene oxide, and also alkylsulphonates, alkyl sulphates, aryl sulphates, tetraalkylammonium halides, trialkylarylammonium halides and alkylamine sulphonates. The dispersants B) can be used individually or else in a mixture. With preference mention may be made of reaction products of caster oil with ethylene oxide in a molar ratio of from 1:20 to 1:60, reaction products of C₆-C₂₀ alcohols with ethylene oxide in a molar ratio of from 1:5 to 1:50, reaction products of fatty amines with ethylene oxide in a molar ratio of from 1:2 to 1:20, reaction products of 1 mol of phenol with 2 to 3 mol of styrene and from 10 to 50 mol of ethylene oxide, reaction products of C₈-C₁₂ alkylphenols with ethylene oxide in a molar ratio of from 1:5 to 1:30, alkylglycosides, C₈-C₁₆ alkylbenzenesulphonic salts, such as calcium salts, monoethanolammonium salts, diethanolammonium salts and triethanolammonium salts, for example.

As examples of nonionic dispersants B) mention may be made of the products known under the names Pluronic® PE 10 100 and Pluronic® F 68 (from BASF) and Atlox® 4913 (from Uniqema). Also suitable are tristyrylphenyl ethoxylates. As examples of anionic dispersants B) mention may be made of the product from Bayer AG known under the name Baykanol® SL (a condensation product of sulphonated ditolyl ether with formaldehyde), which is available commercially, and phosphated or sulphated tristyrylphenol ethoxylates, in which case specific mention may be made of Soprophor® FLK and Soprophor 4D® 384 (from Rhodia).

As dispersants B) mention may also be made by way of example of copolymers of ethylene oxide and propylene oxide, reaction products of tristyrylphenol with ethylene oxide and/or propylene oxide, such as tristyrylphenol ethoxylate containing on average 24 ethylene oxide groups, tristyrylphenol ethoxylate with on average 54 ethylene oxide groups or tristyrylphenol ethoxylate propoxylate with on average 6 ethylene oxide groups and 8 propylene oxide groups, and also phosphated or sulphated tristyrylphenol ethoxylates, such as phosphated tristyrlphenol ethoxylate with an average of 16 ethylene oxide groups, sulphated tristyrylphenol ethoxylate with an average of 16 ethylene oxide groups or ammonium salt of phosphated tristyrylphenol ethoxylate with on average 16 ethylene oxide groups, and also lipoids, such as phospholipid sodium glycolate or lecithin, and also liguinsulphonates. Furthermore, substances having wetting agent properties are also suitable. With preference mention may be made of alkylphenol ethoxylates, dialkylsulphosuccinates, such as sodium diisooctylsulphosuccinate, lauryl ether sulphates, and polyoxyethylene-sorbitan fatty acid esters.

Coating materials E) for the purposes of the invention are, in particular, polyvinyl alcohol, polyvinyl pyrrolidone, saccharides, preferably grape sugars, oligomeric saccharides, especially disaccharides, and with particular preference, cane sugar, or polysaccharides.

Polyvinyl alcohol in the present case refers to both water-soluble polymerization products of vinyl alcohol and to water-soluble, partially hydrolyzed polymers of vinyl acetate. Preference is given to polyvinyl alcohol having an average molecular weight (number average) of from 10,000 to 200,000.

As an example mention may be made of the polyvinyl alcohol product from Clariant known under the tradename Mowiol® 3-83. Preference is also given to a partially hydrolyzed polyvinyl acetate having an average molecular weight (number average) of from 13,000 to 130,000 with an acetate group content of between 1 and 28%.

In the case of Mowiol® 3-83 the stated numbers have the following meanings: 3 describes the viscosity of a 4% strength aqueous solution at 20° C. in mPa.s, 83 indicates the degree of hydrolysis in mol %.

Particularly suitable for use in the present case are polyvinyl alcohols obtained by partial hydrolysis of polyvinyl acetate, having a degree of hydrolysis of from 72 to 99 mol % and a viscosity of from 2 to 40 mPa.s, with particular preference between 3 and 18 mPa.s, measured on a 4% strength aqueous solution at 20° C. Both individual examples of these partially hydrolyzed polyvinyl acetates and also mixtures are suitable.

Suitable additives C) which may be present in the formulations of the invention include penetrants, defoamers, low-temperature stabilizers, preservatives, dyes, redispersants, disintegrants, inert fillers, and film formers.

Suitable penetrants in the present context are all substances which are commonly used to enhance the penetration of active agrochemical substances into plants. Preference is given to alkanol alkoxylates of the formula

R—O—(—AO)ₘH (I)

in which

R is straight-chain or branched alkyl having 4 to 20 carbon atoms,

AO is an ethylene oxide radical, a propylene oxide radical, a butylene oxide radical or mixtures of ethylene oxide and propylene oxide radicals, and

m is a number from 2 to 30.

One particularly preferred group of penetrants are alkanol alkoxylates of the formula

R—O—(—EO—)ₙ—H (Ia)

in which

R is as defined above,

EO is —CH₂—CH₂—O— and

n is a number from 2 to 20.

Another particularly preferred group of penetrants are alkanol alkoxylates of the formula

R—O(—EO—)ₚ—(—PO—)_{q}—H (Ib)

in which

R is as defined above,

EO is —CH₂—CH₂—O—,

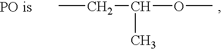

p is a number from 1 to 10 and

q is a number from 1 to 10.

Another particularly preferred group of penetrants are alkanol alkoxylates of the formula

R—O(—PO—)ᵣ—(—EO—)ₛ—H (Ic)

in which

R is as defined above,

EO is —CH₂—CH₂—O—,

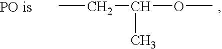

r is a number from 1 to 10 and

s is a number from 1 to 10.

Another particularly preferred group of penetrants are alkanol alkoxylates of the formula

CH₃—(CH₂)ₜ—CH₂—O—(—CH₂—CH₂—O—)ᵤ—H (Id)

in which

t is a number from 8 to 13

and

u is a number from 6 to 17.

In the formulae indicated above

R is preferably butyl, i-butyl, n-pentyl, i-pentyl, neopentyl, n-hexyl, i-hexyl, n-octyl, i-octyl, 2-ethylhexyl, nonyl, i-nonyl, decyl, n-dodecyl, i-dodecyl, lauryl, myristyl, i-tridecyl, trimethylnonyl, palmityl, stearyl, or eicosyl.

An example of an alkanol alkoxylate of the formula (Ic) is 2-ethylhexyl alkoxylate of the formula

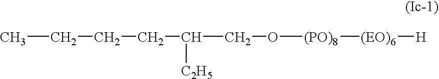

in which

EO is —CH₂—CH₂—O—,

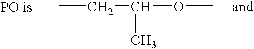

the numbers 8 and 6 represent average values.

Particularly preferred alkanol alkoxylates of the formula (Id) are compounds of this formula in which

t is a number from 9 to 12 and

u is a number from 7 to 9.

A general definition of the alkanol alkoxylates is given by the formulae above. These substances comprise mixtures of substances of the stated type with different chain lengths. The indices therefore come out at average values, which can in some cases deviate from whole numbers.

By way of example, mention may be made of alkanol alkoxylate of the formula (Id) in which

t is the average value 10.5 and

u is the average value 8.4.

The alkanol alkoxylates of the formulae stated are known in principle or can be prepared by known methods (cf. WO 98-35 553 A1, WO 00-35 278 A1 and EP 0 681 865 A1).

Suitable defoamers include all substances which can normally be used for this purpose in agrochemical compositions. Preference is given to silicone oils and magnesium stearate.

Suitable low-temperature stabilizers are all substances which would normally be employed for this purpose in agrochemical compositions. Examples which may be mentioned include urea, glycerol and propylene glycol.

Suitable preservatives include all substances which can normally be used for this purpose in agrochemical compositions of this type. Examples that may be mentioned include Preventol® (from Bayer AG) and Proxel®.

The chemical composition of said commercial products and other mentioned above is:

ATLOX® 4913:

2-Propenoic acid, 2-methyl-, polymer with α-methyl-Ω-hydroxypoly(oxy-1,2-ethanediyl) and methyl-2-methyl-2-propenoate, graft

PLURONIC® 10100:

Oxirane, methyl-, polymer with oxirane, block

PLURONIC® F 68:

Oxirane, methyl-, polymer with oxirane, block

PREVENTOL® D2:

Benzylhemiformal,

PROXEL®GXL:

1,2-Benzisothiazol-3(2H)-one,

SOPROPHOR® 3D384:

Polyethylene glycol mono[tris(α-methylbenzyl)phenyl] ether sulfate, ammonium salt,

SOPROPHOR® FLK:

Polyethylene glycol mono[tris(α-methylbenzyl)phenyl] ether phoshate, potassium salt.

Suitable colorants include all substances which can normally be used for this purpose in agrochemical compositions. Examples that may be mentioned include titanium dioxide, pigment-grade carbon black, zinc oxide and blue pigments and also permanent red FGR.

Suitable redispersants include all substances which can normally be used for this purpose in solid agrochemical compositions. Preference is given to surfactants, swelling agents and sugars. By way of example mention may be made of lactose, urea, polyethylene glycol and tetramethylolpropane.

Suitable disintegrants, as they are known, include substances suitable for accelerating the breakdown of the powder formulations of the invention when combined with water. Preference is given to salts such as sodium chloride and potassium chloride.

Suitable inert fillers include all substances which can normally be used for this purpose, for example, in agrochemical compositions and which do not act as thickeners. Preference is given to inorganic particles, such as carbonates, silicates and oxides, and also to organic substances, such as urea-formaldehyde condensates. Mention may be made by way of example of kaolin, rutile, silica, highly disperse silica, silica gels, and natural and synthetic silicates, and also talc.

Suitable film formers include water-soluble substances normally used for this purpose in active substance formulations. Preference is given to gelatin, water-soluble starch and water-soluble copolymers of polyvinyl alcohol and polyvinyl-pyrrolidone.

In the pulverulent active substance formulations the amount of the individual components can be varied within a relatively wide range. For instance, the concentration

of solid active substances A) is preferably from 10 to 50% by weight, more preferably from 15 to 40% by weight,

of dispersants B) is preferably from 5 to 50% by weight, more preferably from 7.5 to 40% by weight,

of coating material E), in particular of polyvinyl alcohol, is preferably from 10 to 30% by weight, more preferably from 15 to 30% by weight, and

of additives C) is preferably from 0 to 50% by weight, more preferably from 0 to 40% by weight.

The pulverulent active substance formulations of the invention are composed of a multiplicity of individual particles which contain active substance and dispersant and are surrounded by a coat of coating material E), in particular of polyvinyl alcohol. The coat may also comprise other water-soluble, film-forming substances in addition. The particles are in the amorphous state and have an average diameter in the nanometer range. The average particle diameter (number average) of the particles is preferably from 10 to 1000 nm, more preferably from 40 to 500 nm.

The average diameter of the coats of coating material E), in particular of the polyvinyl alcohol coats (i.e. capsules), is preferably from 5 to 500 μm, more preferably from 10 to 150 μm.

In carrying out the process the procedure in step (a) is in particular to suspend finely divided, optionally preground active substance A) and also dispersant B) and, if desired, additives C) in water with stirring. This step is generally operated at a temperature of from 10° C. to 30° C., preferably at room temperature.

The term “finely divided” here means that an average particle size of the active substance A) with a diameter of not more than 50 μm is employed.

In the next step of the process (b), the resulting suspension is admixed under pressure with a compressible fluid D). This step is operated preferably at a pressure of from 50,000 to 500,000 hPa, preferably from 70,000 to 300,000 hPa. The temperature for this operation may correspond to the temperature at which the suspension is prepared in step (a) of the process, or else may be chosen to be higher or lower.

In step (c) of the process the mixture resulting from step (b) is heated until the solid components used, which form the disperse phase, are liquefied and an emulsion is formed in which the components are distributed in the form of droplets in the water phase. This step is generally operated at a temperature below the melting point (under standard conditions) of the respective active substance or, in the case of active substance mixtures, below the melting point of the solid having the highest melting point, preferably at a temperature of from 40° C. to 220° C., more preferably from 50° C. to 220° C. The mixture is preferably heated at a rate such that the emulsion state is present only for a short time. A short time here means, for example, a time in the region of a few milliseconds.

The resultant emulsion (i.e. dispersion of melt in droplet form in the water phase) is first of all homogenized in step (d) of the process of the invention, using for example a jet disperser or other high-pressure homogenizer or a homogenizer operating on the rotor/stator principle, so as to give a fine dispersion. Homogenizing in the homogenizer or jet disperser generally takes place at a temperature of from 40° C. to 220° C.

When carrying out the homogenization in step d) it is normal to operate under a pressure difference in the homogenizer, preferably with a pressure difference of from 40,000 hPa to 1,600,000 hPa, more preferably from 50,000 hPa to 1,000,000 hPa.

Subsequently, the very fine dispersion prepared is admixed in step e), for the purpose of encapsulation, with an aqueous solution of the coating material E), preferably in a concentration of from 10 to 50% by weight, preferably with polyvinyl alcohol, and, where appropriate, with additives C).

In step e) of the process of the invention the dispersion from step d) is depressurized suddenly, causing the compressible fluid D) to expand and to contribute to the destruction (i.e., “explosion”) of the resulting particles into smaller particles, and preferably at the same time is subjected to spray drying with a dry gas, in particular with dry air or inert gas, more preferably with nitrogen or a noble gas.

For the spray drying the temperature can be varied within a relatively wide range. It is preferred to operate at a dry gas entry temperature of from 100° C. to 200° C., more preferably from 120° C. to 180° C., and at a dry gas exit temperature of from 50° C. to 100° C., more preferably from 60° C. to 90° C.

In one particularly preferred variant of the process it is possible to use freeze drying to remove the water present in the emulsion from step d). This method is appropriately employed when the active substances are unstable at relatively high temperatures.

Both spray drying and freeze drying are preferably operated so that only a very low level of residual moisture remains in the powder formulation. Drying is generally taken to a point where the residual moisture content lies below 1% by weight. Residual moisture content here denotes the amount of volatile compounds such as water or, where appropriate, solvents.

If polyvinyl alcohol is added as additive C) as early as in step a) when the process of the invention is being carried out, there is no need to add this capsule-forming coating material E) in step d).

The process of the invention can be carried out either continuously or batchwise.

For carrying out the process of the invention it is preferred to use a novel apparatus, at least comprising

a pump capable of introducing a compressible fluid D) under pressure into

a pressure-resistant vessel provided with a stirrer and connected via

a pump suitable for generating pressure to

a heat exchanger,

to which there is connected a jet disperser or homogenizer, from which

a pipeline closable with a valve leads back into the vessel and from which a pipeline is connected to

a metering pump and also, where appropriate, to a mixing vessel, with the pipeline which leads on from the latter

being connected to a spray dryer.

### BRIEF DESCRIPTION OF THE DRAWING

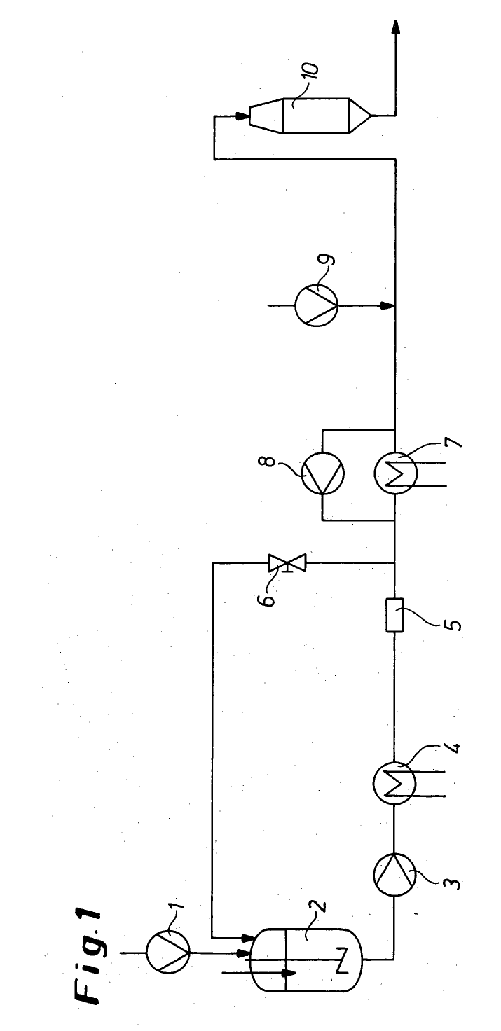

A diagram of an apparatus suitable for carrying out the process of the invention is shown in FIG. 1. In this FIGURE

1=metering pump for compressible fluid D)

2=pressure-resistant vessel provided with stirrer

3=pump suitable for generating pressure

4=heat exchanger

5=jet disperser

6=valve

7=condenser in cooling circuit

8=pump installed in the cooling circuit

9=metering pump for supplying solution

10=spray dryer

The heat exchanger 4 is an apparatus which allows rapid heating of the incoming suspension to the desired temperature.

The jet disperser 5 is constructed so that the incoming emulsion is dispersed through a nozzle. The fineness of the dispersion produced is dependent on the homogenizing pressure and on the nozzle used. The smaller the nozzle bore, the finer the dispersion obtained. Generally speaking, nozzles are used whose bores are between 0.1 and 1 mm, preferably between 0.2 and 0.7 mm.

The pump 9 is a metering device which is attached to the pipeline leading away from the cooling circuit. At this point in the apparatus it is also possible to install an additional mixing vessel with stirrer.

The spray dryer 10 is a device of this type which is such that the dispersion can be depressurized and, consequently, the compressible fluid D) expands, and such that the incoming aqueous solution can have its water removed. The spray dryer may also be replaced by a freeze dryer.

Carrying out the process of the invention using the apparatus stated involves in particular, in the first step, suspending one or more solid active substances A) with a crystalline structure and also, where appropriate, additives C) in a finely divided state in the vessel 2 in a mixture of water and dispersant B). The components can be combined in a preground state. An alternative possibility, however, is to comminute the constituents after they have been mixed, using a rotor/stator disperser, a colloid mill or a bead mill.

Using the pump 1, a compressible fluid D) is added under pressure to the suspension thus prepared in the vessel 2.

The dispersion prepared in this way is conveyed by the pump 3 via the heat exchanger 4 into the downstream jet disperser 5. In addition to conveying, the pump also has the function of building up the necessary dispersing pressure.

Before entering the jet disperser 5 the dispersion in the heat exchanger 4 is rapidly heated to a temperature above the melting point of the solid phase, so briefly forming an emulsion. This emulsion is then finely homogenized in the jet disperser 5 and immediately after its passage through the jet disperser is cooled in the cooling circuit system 7/8. To minimize the cooling time, the dispersion is passed into the condenser 7 and recirculated using the pump 8 with a circulation flow rate, that is approximately ten times the flow rate of the emulsion leaving the disperser. As a result of the cooling loop, quenching ensures that the emulsion is cooled within a period of milliseconds and that the particles of active substance solidify amorphously.

Following passage through the cooling circuit, the metering pump 9 adds an aqueous solution of polyvinyl alcohol and also, where appropriate, further coating materials and/or other additives C) to the dispersion of amorphous particles. In one particular variant of the process, however, it is also possible for the polyvinyl alcohol solution used to encapsulate the amorphous particles to be added as early as in the cooling circuit 7/8 or even in the batching vessel 2.

Immediately thereafter the solution prestabilized in this way is introduced into the spray dryer 10 and depressurized; here, the compressible fluid D) expands, the water is removed from the dispersion, and the particles of active substance are encapsulated by coating material.

The resulting product in each case is a free-flowing powder. The particle size depends in the manner indicated on the nozzle-spray conditions in the jet disperser.

In one variant of the process a portion of the flow is removed between the jet disperser 5 and the cooling circuit 7/8 and is recycled by the valve 6 to the vessel 2. As a result of this circulation regime it is possible to homogenize the dispersion through the jet disperser a number of times. In circulation mode, the dispersion can also be temperature-conditioned by way of the vessel 2 instead of by the heat exchanger 4.

The powder formulations of the invention are stable even when stored for a relatively long period of time. They can be converted to homogeneous spray liquids by stirring them into water. Within the area of application these spray liquids are used by common, known methods, i.e., for example, by spraying, pouring or injection. A further option is to granulate the powders or to process them to tablets, pastes or other application forms.

The application rate of the powder formulations of the invention can be varied within a relatively wide range. The rate is guided by the active substances A) present in each case and by the amount thereof in the formulations.

By means of the powder formulations of the invention it is possible to deliver active substances A) in a particularly advantageous way. The active substances contained are readily bioavailable and develop a biological activity which is substantially better than that of those conventional formulations in which the active components are present in the crystalline state.

The invention is illustrated below by way of example, with reference to FIG. 1.

### EXAMPLE 1

In a vessel 2, 3 liters of a suspension consisting of

5 parts by weight of the active substance of the formula

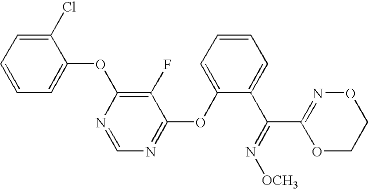

5 parts by weight of emulsifier (phosphoric acid mono-/diester mixture of a tristyrylphenol ethoxylate containing on average 16 ethylene oxide units, tradename Soprophor® 3D33)

90 parts by weight of water

were mixed and, using a rotor/stator system, were dispersed so that the particle size was between 1 and 10 μm. In the pressure vessel 2, 600 g of CO₂ were pumped into the suspension thus prepared. With a circulation flow of 180 l/h, a system pressure of 54,000 hPa and temperature conditioning at 70° C., the dispersion was conveyed by means of pump 3 under a homogenizing pressure of 50,000 hPa through a jet disperser 5 with a nozzle bore of 0.2 mm. After about 5 pumped circulation cycles the pump 3 was shut off and the contents of the vessel were mixed with 1.2 liters of a 25% strength by weight solution of polyvinyl alcohol (Mowiol® 3-83 from Clariant) in water, from line 9, and immediately thereafter the mixture was depressurized through a hollow-cone nozzle into a spray dryer 10, in the course of which the CO₂ escaped, and dried at an outgoing air temperature of <80° C. This gave a free-flowing powder with a particle size of approximately 20 μm, which it was possible to redisperse in water. The size of the active substance particles in the dispersion was 0.2 μm. The particle morphology was amorphous.

### EXAMPLE 2

In a vessel 2 2.8 kg of a suspension consisting of

5.4 parts by weight of the active substance ibuprofen

5.4 parts by weight of emulsifier (Tween 80),

21.6 parts by weight of capsule material (25% strength by weight solution of polyvinyl alcohol Mowiol® 3-83 from Clariant in water),

67.6 parts by weight of water

were mixed and, using a rotor/stator system, were dispersed so that the particle size was between 1 and 10 μm. In the pressure vessel 2 470 g of CO₂ were pumped into the suspension thus prepared. With a circulation flow of 120 kg/h, a system pressure of 80,000 hPa, set using nitrogen, and temperature conditioning at 60° C. the dispersion was conveyed under a homogenizing pressure of 50,000 hPa through a jet disperser 5 having a nozzle bore of 0.2 mm. After about 20 pump circulation cycles the dispersion was depressurized through a hollow-cone nozzle into a spray dryer 10, in the course of which the CO₂ escaped, and dried at an outgoing air temperature of <80° C. This gave a free-flowing powder which it was possible to redisperse in water. The particle size of the dispersion was 0.2 μm. The particle morphology was amorphous. Drying the powder retained the morphology.

We claim:

1. Pulverulent active substance formulations comprised of
particles of at least one active substance A) which is solid at room temperature,
at least one dispersant B),
a coating material E), and
optionally additives C),
wherein the individual active substance particles are in an amorphous state, have an average diameter of not more than 1 μm, and are coated with coating material E.

2. Active substance formulations according to claim 1, wherein active substance A) is selected from the group consisting of ibuprofen, clotrimazole, fluconazole, indoxacarb, acetylsalicylic acid and ciprofloxazin.

3. Active substance formulations according to claim 1, wherein active substance A) is selected from the group consisting of fungicides, bactericides, insecticides, acaricides, nematicides, molluscicides, herbicides and plant growth regulators.

4. Active substance formulations according to claim 1, wherein active substance A) is selected from the group consisting of vitamins, carotenoids and flavors.

5. Active substance formulations according to claim 1 wherein dispersants B) are selected from the group consisting of nonionogenic, anionic, cationic and zwitterionic substances having surface-active properties.

6. Active substance formulations according to claim 1, comprising additives C) wherein additives C) are selected from the group consisting of penetrants, defoamers, low-temperature stabilizers, preservatives, dyes, redispersants, disintegrants, inert fillers and film formers.

7. Active substance formulations according to claim 1, wherein coating material E) is selected from the group consisting of polyvinyl alcohol, polyvinyl-pyrrolidone and saccharides.

8. Active substance formulations according to claim 7, wherein said coating material E is a saccharide and said saccharide is grape sugar, cane sugar or a polysaccharide.

9. Active substance formulations according to claim 1, comprising
from 10 to 50% by weight active substances A),
from 5 to 50% by weight dispersant B)
from 10 to 30% by weight coating material E), and
from 0 to 50% by weight additives C).

10. Active substance formulation according to claim 7 or 9 wherein said coating material E) is polyvinyl alcohol.

11. Active substance formulation according to claim 9, wherein said amount of active substance A) is from 15 to 40% by weight, said amount of dispersant B) is from 7.5 to 40% by weight, the amount of said coating material E) is from 15 to 30% by weight and the amount of said additives C) is from 0 to 40% by weight.

12. Active substance formulations according to claim 1, wherein said particles have a number average diameter of from 10 to 1,000 nm.

13. Active substance formulations according to claim 12, wherein said number average diameter is from 40 to 500 nm.

14. Active substance formulations according to claim 1, wherein said coating material E) is polyvinul alcohol, said active substance formulations are in the form of capsules of said polyvinyl alcohol and said capsules have an average capsule diameter of from 50 to 500 μm.

15. Active substance formulations according to claim 14, wherein said average capsule diameter is from 10 to 150 μm.

16. Process for producing the pulverulent active substance formulations of claim 1, which comprises
a) suspending at least one active substance A) which is solid at room temperature, at least one dispersant B), and optionally additives C) in an aqueous phase,
b) adding at least one compressible fluid D) in the supercritical state under pressure to the suspension formed in a),
c) heating the mixture formed in b) until the solid components are liquefied to form a dispersion,
d) homogenizing the dispersion and then adding an aqueous solution of coating material E), and optionally additives C),
e) depressurizing the dispersion and subjecting it to drying.

17. Process according to claim 16, wherein step a) is carried out at a temperature of from 10° C. to 30° C.

18. Process according to claim 16 or 17, wherein step b) is carried out at a pressure of from 50,000 to 500,000 hPa.

19. Process of claim 18, wherein said pressure is from 70,000 to 300,000 hPa.

20. Process according to one of claims 16 or 17, wherein step c) is carried out at a temperature below the melting point (under standard conditions) of the respective active substance or, in the case of active substance mixtures, below the melting point of the solid having the highest melting point.

21. Process according to claim 20, wherein said respective active substance or said active substance having the highest melting point has a melting point of from 40° C. to 220° C.

22. Process according to claim 21, wherein said melting point is from 50° C. to 220° C.

23. Process according to claim 16 or 17, wherein in step d) the emulsion is homogenized using a jet disperser or other high-pressure homogenizer or a homogenizer operating on the rotor/stator principle.

24. Process according to claim 23, wherein step d) is conducted with a homogenizer subjecting the dispersion to a pressure difference in the homogenizer at from 40,000 hPa to 1,600,000 hPa.

25. Process according to claim 24, wherein said pressure difference is from 50,000 hPa to 1,000,000 hPa.

26. Process for producing the pulverulent active substance formulations of claim 1, comprising the steps of
a) suspending at least one active substance A) which is solid at room temperature, at least one dispersant B), and optionally additives C) in an aqueous phase,
b) adding at least one compressible fluid D) in the supercritical state under pressure to the suspension formed in a),
c) heating the mixture formed in b) until the solid components it comprises are liquefied,
d) homogenizing the resultant dispersion,
e) mixing the homogenized dispersion with an aqueous solution of coating material E) in a concentration of from 10 to 50% by weight, and optionally with additives C) and then depressurizing the dispersion and subjecting it to drying.

27. Process according to claim 16, 17 or 26, wherein said drying comprises spray drying or freeze drying.

28. Process according to claim 27, wherein said drying is spray drying.

29. Process according to claim 16, wherein in step e) the dispersion from step d) is depressurized at a rate sufficient to expand the compressible fluid and thereby explode the dispersed solid components, and optionally, at the same time is subjected to spray drying with a dry gas.

30. The process of claim 29, wherein said dry gas is dry air, nitrogen or a noble gas.

31. The process of claim 30, wherein said dry gas is a noble gas.

32. Process according to claim 29, wherein said gas is subjected to spray drying in step e), and said spray drying in step e) is carried out at a dry gas entry temperature of from 100° C. to 200° C., and a dry gas exit temperature of from 50° C. to 100° C.

33. Process of claim 32, wherein said spray drying is carried out at a temperature of from 120° C. to 180° C., and said dry gas exit temperature is from 60° C. to 90° C.

34. Process according to claim 16, wherein in step e) the water present in the homogenized dispersion from step d) is removed by freeze drying.

35. Process according to claim 16, wherein said compressible fluid D) is selected from the group consisting of hydrocarbons having 1 to 6 carbon atoms, Freons, nitrogen, noble gases, gaseous oxides, ammonia, alcohols having 1 to 4 carbon atoms, halogenated hydrocarbons, and mixtures thereof.

36. Process of claim 35, wherein said compressible fluid D) is selected from the group consisting of methane, ethane, propane, butane, pentane, n-hexane, i-hexane, nitrogen, noble gases, N₂O, CO₂, ammonia, methanol, ethanol, isopropanol, n-propanol, butanol, halogenated hydrocarbons, and mixtures thereof.

37. Method for applying an active substance to a site, which comprises applying said active substance to said site in the form of a pulverulent active substance formulation of claim 1.

38. Method of applying active substances to a targeted organism and/or its habitat, which comprises delivering said active substances to said organism or habitat in the form of the pulverulent active substance formulation of claim 1, optionally after dilution with extenders and/or surface-active substances.

39. Apparatus for carrying out the process according to claim 16, comprising at least a device for metering a compressible fluid D) under pressure into a pressure-resistant vessel (2) which is provided with a stirrer and is connected via a pump (3) suitable for generating pressure to a heat exchanger (4), to which a homogenizing jet disperser (5), is connected, from which a pipeline closable with a valve leads back into the vessel (2) and from which, optionally, a pipeline leads to a cooling circuit which is provided with a pump and whose outlet line is connected to a metering pump (9) and also, optionally, to a mixing vessel, the pipeline leading on from the latter being connected to a spray dryer (10).

40. Process for producing the pulverulent active substance formulations of claim 1, comprising the steps of
a) suspending at least one active substance A) which is solid at room temperature, at least one dispersant B), at least one coating material E), and optionally additives C) in an aqueous phase,
b) adding at least one compressible fluid D) in the supercritical state under pressure to the suspension formed in a),
c) heating the mixture formed in b) until the solid components it comprises are liquefied,
d) homogenizing the resultant dispersion, and then
e) depressurizing the dispersion and subjecting it to drying.
